(19) United States
(12) Patent Application Publication
(10) Pub. No.: US 2004/0092491 A1
(43) Pub. Date: May 13, 2004

(54) Method of treating sepsis-induced ARDS

(75) Inventors: Gary Nieman, Manlius, NY (US); Sanford R. Simon, Stony Brook, NY (US); Lorne M. Golub, Smithtown, NY (US); Hsi-Ming Lee, Setauket, NY (US); Jay Steinberg, Syracuse, NY (US); Henry Schiller, Rochester, MN (US); Jeff Halter, Liverpool, NY (US); Anthony Picone, Manlius, NY (US); William Marx, Jamesville, NY (US); Louis Gatto, Cortland, NY (US); Charles Lutz, Fayetteville, NY (US)

Correspondence Address: HOFFMANN & BARON, LLP, 6900 JERICHO TURNPIKE, SYOSSET, NY, 11791, US

(73) Assignee: The Research Foundation of State University of New York

(21) Appl. No.: 10/291,194

(22) Filed: Nov. 9, 2002

(51) Int. Cl. A61K 31/65

(52) U.S. Cl. 514/152

(57) ABSTRACT

The invention is method for preventing sepsis-induced ARDS in a mammal in need thereof, the method comprises administering to the mammal a tetracycline compound in an amount that is effective to prevent sepsis-induced ARDS but has substantially no antibiotic activity.

### BACKGROUND OF THE INVENTION

Acute respiratory distress syndrome (ARDS) is a critical illness characterized by acute lung injury leading to permeability pulmonary edema and respiratory failure. Despite significant advances in critical care management, mortality from ARDS remains at 40-60%. Each year over 100,000 people die in the United States from complications of ARDS. Current treatment is predominantly support of the respiratory system with, for example, mechanical ventilation.

In general, the development of ARDS can be separated into two phases: an initiator stage followed by an effector stage. The initiator phase of ARDS involves the release of inflammatory mediators (i.e. cytokines; complement and coagulation factors; and arachidonic acid metabolites) which promote systemic inflammation resulting in pulmonary neutrophil sequestration. The second stage, the effector phase, involves the activation of neutrophils with subsequent release of toxic oxygen radicals and proteolytic enzymes, specifically neutrophil elastase (NE). Neutrophil elastase has the capacity to injure pulmonary endothelial cells and degrade products of the extracellular matrix, such as elastin, collagen, and fibronectin which comprise the lung basement membrane.

Many diverse forms of ARDS exist with disparate etiologies and courses, although the end-state pathologies of these diverse forms are the same. Examples of clinical events that may precipitate different forms of ARDS include trauma, hemorrhage, diffuse pneumonia, inhalation of toxic gases, and sepsis. Each of these forms of ARDS differs in its kinetics and development. For example, the timing of initiator and effector stages may differ; or the levels of various inflammatory mediators or neutrophils may differ. Different forms of ARDS demand different treatment strategies.

For example, in trauma-induced ARDS, an injury to the endothelium, epithelium or internal organs activates neutrophils at the site of the injury. These neutrophils then sequester in the intrapulmonary area, and are activated further. A method for preventing this form of ARDS has been disclosed in U.S. Pat. No. 5,877,091. In this method, tetracycline compounds are administered prior to significant intrapulmonary accumulation of neutrophils.

An example of one of the most clinically significant forms of ARDS is sepsis-induced ARDS. Sepsis is the overwhelming systemic response to infection of the blood. Any viable microbe, including bacteria, fungi and viruses, can be the source of the infection. As the course of the sepsis proceeds, ARDS may be induced.

Another form of ARDS, endotoxin-induced ARDS, is rarely seen clinically. In this form of ARDS, endotoxin, i.e. lipopolysaccharide (LPS), is released into the body at a high rate. The source of the LPS is gram negative bacteria that has been disrupted.

LPS induces a syndrome which resembles sepsis, i.e. endotoxemia. LPS activates the neutrophils which subsequently sequester in the lung and ARDS ensues. One of the rare clinical scenarios which may precipitate endotoxin-induced ARDS involves patients whose gram negative bacterial infections were treated with antibiotics. The antibiotic disrupts the bacteria, thus allowing the endotoxin to be released into the body.

Experimental animal models which replicate endotoxin-induced ARDS (“the LPS model”) have been used by many researchers. These models include the infusion of LPS into animals.

For example, Japanese patent application No. WO95/03057 of Chugai Pharmaceuticals discloses an experimental model that includes the injection of LPS into mice. It is stated that this model replicates conditions caused by endotoxins, such as ARDS. The treatment disclosed by Chugai for such conditions is an endotoxin neutralizer which contains, as an active ingredient, a tetracycline or its derivative.

Also, Sakamaki et al., Effect of a Specific Neutrophil Elastase Inhibitor, ONO-5046, on Endotoxin-Induced Acute Lung Injury, Am. J. Respir. Crit. Care Med. 153, 391-397 (1996), disclose a guinea pig model of acute lung injury induced by LPS. The authors report that the neutrophil-elastase inhibitor, N-[2-[4-(2,2-dimethylpropionyloxy)-phenylsulfonylamino]benzoyl]aminoacetic acid (ONO-5046), inhibits neutrophil elastase activity.

Recently, the LPS model has been used to evaluate certain types of immunotherapeutic agents. These immunotherapeutic agents block the activity of particular cytokines involved in the initiator phases of sepsis and ARDS. The animals used in the LPS models responded dramatically well. The successful immunotherapeutic agents were then transferred to clinical settings to treat patients suffering from sepsis and sepsis-induced ARDS. However, despite the preclinical successes, human clinical trials did not demonstrate any improvement in patient survival.

Remick et al. postulated that the syndrome induced by LPS differs substantially from the clinically relevant sepsis which is induced by intact bacteria (Shock 13(2): 110-6 (2000)). They directly compared the mortality, morbidity, and immunopathology resulting from the LPS model with those resulting from the cecal ligation and puncture model (“the CLP model”). Unlike the LPS model which only introduces endotoxin into a system, the CLP model introduces intact bacteria. Remick et al. observed that the LPS and the CLP models result in similar mortality levels; but these models significantly differ in their kinetics and cytokine production. They concluded that the LPS model does not adequately reproduce the complex pathology, such as the cytokine profile, of the clinically relevant sepsis. It follows that the ARDS which ensues from LPS injection is different from the ARDS which ensues from intact bacteria administration.

Thus, endotoxin-induced ARDS differs substantially in both etiology and immunopathology from the clinically relevant sepsis-induced ARDS. Accordingly, the endotoxin-induced ARDS, in particular, the LPS model of ARDS, does not teach a skilled artisan anything about the clinically relevant sepsis-induced ARDS. In particular, the teaching that neutrophil elastase and endotoxin inhibitors are useful for treating endotoxin-induced ARDS would not have taught a skilled artisan how to treat sepsis-induced ARDS. Whether these inhibitors would be effective to treat sepsis-induced ARDS would not have been predictable.

The prior art treatments for ARDS discussed above are inadequate. ARDS-induced by sepsis remains a common cause of death in intensive care units in the United States, and its incidence is rising. This growth is most likely due to the increased use of invasive devices and immunosuppressive therapies, higher numbers of immunocompromised patients, and increasing antibiotic resistance. Accordingly, there is an urgent need for an effective treatment of clinically relevant sepsis-induced ARDS.

### SUMMARY OF THE INVENTION

The present invention provides a method for preventing sepsis-induced ARDS in a mammal in need thereof. The method comprises administering to the mammal a tetracycline compound in an amount that is effective to prevent sepsis-induced ARDS, but has substantially no antibiotic activity.

### BRIEF DESCRIPTION OF THE FIGURES

FIG. 1. Seven day survival rate in rats from all treatment groups. A significant improvement in survival is seen after single dose administration of COL-3 [CLP+COL-3 (SD); p<0.05 vs CLP+CMC]. An enhanced survival benefit is noted with a repeat dose of COL-3 at 24 hours post CLP [CLP+COL-3 (MD); p<0.05 vs both CLP+CMC and CLP+COL-3 (SD)].

FIG. 2. Quantification of lung tissue levels of MMP-2 by immunohistochemistry. Note a significant increase in alveolar MMP-2 levels in the CLP+CMC group as compared to all other groups. A single dose of COL-3 [CLP+COL-3 (SD)]significantly reduced MMP-2 levels from the CLP+CMC group. A further reduction in MMP-2 levels were noted in the CLP+COL-3 (MD) group as compared to both the CLP+CMC and CLP+COL-3 (SD) groups. Data are mean±SE, *=p<0.05 vs all other groups.

FIG. 3. Quantification of lung tissue levels of MMP-9 by immunohistochemistry. Note a significant increase in alveolar MMP-9 levels in the CLP+CMC group as compared to the CLP+COL-3 (MD) and both Sham groups. A single dose of COL-3 [CLP+COL-3 (SD)] reduced MMP-9 levels compared to the CLP+CMC group, but was not statistically significant. Multiple doses of COL-3 [CLP+COL-3 (MD)] significantly reduced MMP-9 levels as compared to both the CLP+CMC and CLP+COL-3 (SD) groups. Data are mean±SE, *=p<0.05 vs CLP+COL-3 (MD) and both Sham groups.

FIG. 4. Pulmonary edema as assessed by gravimetric lung water measurement (W/D weight ratio). Note a significant increase in lung water in the CLP+CMC group as compared to all other groups. A single dose of COL-3 [CLP+COL-3 (SD)] significantly reduced lung water as compared to the CLP+CMC group. Lung water was further reduced with repeat dosing of COL-3 [CLP+COL-3 (MD)]. Data are mean±SE, *=p<0.05 vs all other groups.

FIG. 5. Serum COL-3 concentration at 48 hours post CLP in all groups. Note a significant elevation in COL-3 concentration in the CLP+COL-3 (MD) group as compared to all other groups. Data are mean±SE, *=p<0.05 vs. all other groups.

FIG. 6. Correlation between an increase in COL-3 concentration and improved survival. Data points represent individual animals, p<0.02.

FIG. 7. Correlation between an increase in COL-3 concentration and a decrease in MMP-2 levels. Data points represent individual animals, p<0.008.

FIG. 8. Correlation between a reduction in MMP-2 levels and improved survival. Data points represent individual animals, p<0.03.

FIG. 9. Correlation between a reduction in MMP-9 levels and improved survival. Data points represent individual animals, p<0.0001.

FIG. 10. PaO₂/FiO₂ results for Control Group, SMA+FC Group and SMA+FC+COL-3 Group.

FIG. 11. Pulmonary Edema results for Control Group, SMA+FC Group and SMA+FC+COL-3 Group.

FIG. 12. Gross photographs of lungs from an animal in the SMA+FC+COL-3 Group and the SMA+FC Group.

### DETAILED DESCRIPTION OF THE INVENTION

The present invention provides a method for preventing sepsis-induced acute respiratory distress syndrome, i.e. sepsis-induced ARDS, in a mammal. As used herein, the term “sepsis-induced ARDS” is an ARDS which was precipitated by a clinically relevant sepsis.

Sepsis is the overwhelming systemic response to infection of the blood. A clinically relevant sepsis is a sepsis in which the source of the infection is any viable, intact microbe, including bacteria, fungi and viruses. A clinically relevant sepsis cannot be replicated in the body by the administration of endotoxin alone. Once the course of the sepsis has proceeded to a certain point, ARDS results.

ARDS is the rapid onset of progressive malfunction of the lungs. The condition is associated with extensive lung inflammation and the accumulation of fluid in the air sacs leading to the inability of the lungs to take up oxygen. ARDS is also referred to as adult respiratory distress syndrome.

A mammal which can benefit from the treatment prescribed by the instant invention could be any mammal. Categories of mammals include humans, farm mammals, domestic mammals, laboratory mammals, etc. Some examples of farm mammals include cows, pigs, horses, goats, etc. Some examples of domestic mammals include dogs, cats, etc. Some examples of laboratory mammals include rats, mice, rabbits, guinea pigs, etc.

For the purposes of the instant specification, sepsis-induced ARDS is considered to be prevented if the tetracycline leads to a significant inhibition of the pulmonary injury. As a result of the treatment, a patient would not sustain any pulmonary injury, or would sustain significantly less pulmonary injury, than without the treatment. In other words, the patient would have an improved medical condition as a result of the treatment.

The method of the invention involves administration of a tetracycline compound of the invention any time before the onset of ARDS. For the purposes of this specification, the onset of ARDS in mammal is the time when three particular pulmonary events occur simultaneously while the pulmonary wedge pressure remains in the normal range. These three pulmonary events are: i) a significantly low PaO₂/FiO₂ ratio; ii) a significant bilateral interstitial pulmonary infiltration; and iii) the onset of the clinical symptoms of ARDS.

The PaO₂ is the partial pressure of oxygen in the plasma phase of arterial blood. The FiO₂ is the fraction of inspired oxygen. A significantly low PaO₂/FiO₂ ratio is a value which is below approximately 250.

A significant bilateral interstitial pulmonary infiltration can be seen in a chest x-ray. A person skilled in the art would be able to determine whether the infiltration is to be considered significant.

The clinical symptoms of ARDS include refractory hypoxemia and poor respiratory compliance.

The pulmonary wedge pressure is considered to be in the normal range below approximately 18 mmHg.

Preferably, a tetracycline compound is administered any time after the onset of systemic inflammatory response syndrome (SIRS) and before the onset of ARDS. SIRS is a systemic inflammatory response. The onset of SIRS is considered to have occurred if two or more of the following clinical symptoms appear: Temperature>38° C. or <36° C.; Heart rate >90 beats/min; Respiratory rate >20 breaths/min or PaCO₂<32 mm Hg and WBC count >12,000/mm³, or <4000/mm³. Preferably, a tetracycline compound is administered at the first appearance of SIRS.

The amount of a tetracycline compound administered to a mammal in accordance with the present invention is an amount which is effective for its purpose i.e. preventing sepsis-induced ARDS, but which has substantially no antibiotic activity.

The tetracycline compound can be an antibiotic or non-antibiotic compound. The tetracyclines are a class of compounds of which tetracycline is the parent compound. Tetracycline has the following general structure:

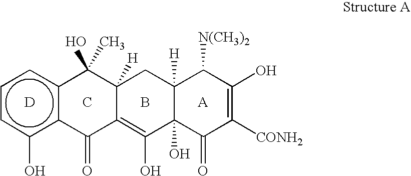

The numbering system of the multiple ring nucleus is as follows:

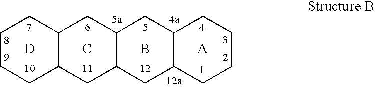

Tetracycline, as well as the 5-hydroxy (oxytetracycline, e.g. Terramycin) and 7-chloro (chlorotetracycline, e.g. Aureomycin) derivatives, exist in nature, and are all well known antibiotics. Semisynthetic derivatives such as 7-dimethylaminotetracycline (minocycline) and 6α-deoxy-5-hydroxytetracycline (doxycycline) are also known tetracycline antibiotics. Natural tetracyclines may be modified without losing their antibiotic properties, although certain elements of the structure must be retained to do so.

Some examples of antibiotic (i.e. antimicrobial) tetracycline compounds include doxycycline, minocycline, tetracycline, oxytetracycline, chlortetracycline, demeclocycline, lymecycline and their pharmaceutically acceptable salts. Doxycycline is preferably administered as its hyclate salt or as a hydrate, preferably monohydrate.

Non-antibiotic tetracycline compounds are structurally related to the antibiotic tetracyclines, but have had their antibiotic activity substantially or completely eliminated by chemical modification. For example, non-antibiotic tetracycline compounds are capable of achieving antibiotic activity comparable to that of tetracycline or doxycycline at concentrations at least about ten times, preferably at least about twenty five times, greater than that of tetracycline or doxycycline, respectively.

Examples of chemically modified non-antibiotic tetracyclines (CMTs) include 4-de(dimethylamino)tetracycline (CMT-1), tetracyclinonitrile (CMT-2), 6-demethyl-6-deoxy-4-de(dimethylamino)tetracycline (CMT-3), 7-chloro-4-de(dimethylamino)tetracycline (CMT-4), tetracycline pyrazole (CMT-5), 4-hydroxy-4-de(dimethylamino)tetracycline (CMT-6), 4-de(dimethylamino-12α-2deoxytetracycline (CMT-7), 6-deoxy-5α-hydroxy-4-de(dimethylamino)tetracycline (CMT-8), 4-de(dimethylamino)-12α-deoxyanhydrotetracycline (CMT-9), 4-de(dimethylamino)minocycline (CMT-10).

Tetracycline derivatives, for purposes of the invention, may be any tetracycline derivative, including those compounds disclosed generically or specifically in co-pending U.S. patent application Ser. Nos. 09/573,654 filed on May 18, 2000 and 10/274,841 filed on Oct. 18, 2002, which are herein incorporated by reference.

The minimal amount of the tetracycline compound administered to a human is the lowest amount capable of providing effective treatment of sepsis-induced ARDS. Effective treatment is a prevention or inhibition of ARDS. The amount of the tetracycline compound is such that it does not significantly prevent the growth of microbes, e.g. bacteria.

There are two manners in which to describe the administered amount of a tetracycline compound, by daily dose and by serum level.

Tetracycline compounds that have significant antibiotic activity may, for example, be administered in a dose (measured either by daily dose or serum level) which is 10-80% of the antibiotic dose. More preferably, the antibiotic tetracycline compound is administered in a dose which is 40-70% of the antibiotic dose.

Antibiotic daily doses are known in art. Some examples of antibiotic doses of members of the tetracycline family include 50, 75, and 100 mg/day of doxycycline; 50, 75, 100, and 200 mg/day of minocycline; 250 mg of tetracycline one, two, three, or four times a day; 1000 mg/day of oxytetracycline; 600 mg/day of demeclocycline; and 600 mg/day of lymecycline.

Examples of the maximum non-antibiotic doses of tetracyclines based on steady-state pharmacokinetics are as follows: 20 mg/twice a day for doxycycline; 38 mg of minocycline one, two, three or four times a day; and 60 mg of tetracycline one, two, three or four times a day.

In a preferred embodiment, doxycycline is administered in a daily amount of from about 30 to about 60 milligrams, but maintains a concentration in human plasma below the threshold for a significant antibiotic effect.

In an especially preferred embodiment, doxycycline hyclate is administered at a 20 milligram dose twice daily. Such a formulation is sold for the treatment of periodontal disease by CollaGenex Pharmaceuticals, Inc. of Newtown, Pa. under the trademark Periostat®.

The administered amount of a tetracycline compound described by serum levels follows.

Some examples of the approximate antibiotic serum concentrations of members of the tetracycline family follow. A single dose of two 100 mg minocycline HCl tablets administered to adult humans results in minocycline serum levels ranging from approximately 0.74 to 4.45 μg/ml over a period of an hour. The average level is 2.24 μg/ml.

Two hundred and fifty milligrams of tetracycline HCl administered every six hours over a twenty-four hour period produces a peak plasma concentration of approximately 3 μg/ml. Five hundred milligrams of tetracycline HCl administered every six hours over a twenty-four hour period produces a serum concentration level of approximately 4 to 5 μg/ml.

In one embodiment, the tetracycline compound can be administered in an amount which results in a serum concentration between about 0.1 and 10.0 μg/ml, more preferably between 0.3 and 5.0 μg/ml. For example, doxycycline is administered in an amount which results in a serum concentration between about 0.1 and 0.8 μg/ml, more preferably between 0.4 and 0.7 μg/ml.

Some examples of the plasma antibiotic threshold levels of tetracyclines based on steady-state pharmacokinetics are as follows: 1.0 μg/ml for doxycycline; 0.8 μg/ml for minocycline; and 0.5 μg/ml for tetracycline.

Non-antibiotic tetracycline compounds can be used in higher amounts than antibiotic tetracyclines, while avoiding the indiscriminate killing of microbes, and the emergence of resistant microbes. For example, 6-demethyl-6-deoxy-4-de(dimethylamino)tetracycline (CMT-3) may be administered in doses of about 40 to about 200 mg/day, or in amounts that result in serum levels of about 1.55 μg/ml to about 10 μg/ml.

The actual preferred amounts of tetracycline compounds in a specified case will vary according to the particular compositions formulated, the mode of application, the particular sites of application, and the subject being treated (e.g. age, gender, size, tolerance to drug, etc.)

The tetracycline compounds can be in the form of pharmaceutically acceptable salts of the compounds. The term “pharmaceutically acceptable salt” refers to a salt prepared from tetracycline compounds and pharmaceutically acceptable non-toxic acids or bases. The acids may be inorganic or organic acids of tetracycline compounds. Examples of inorganic acids include hydrochloric, hydrobromic, nitric hydroiodic, sulfuric, and phosphoric acids. Examples of organic acids include carboxylic and sulfonic acids. The radical of the organic acids may be aliphatic or aromatic. Some examples of organic acids include formic, acetic, phenylacetic, propionic, succinic, glycolic, glucuronic, maleic, furoic, glutamic, benzoic, anthranilic, salicylic, phenylacetic, mandelic, embonic (pamoic), methanesulfonic, ethanesulfonic, panthenoic, benzenesulfonic, stearic, sulfanilic, alginic, tartaric, citric, gluconic, gulonic, arylsulfonic, and galacturonic acids. Appropriate organic bases may be selected, for example, from N,N-dibenzylethylenediamine, chloroprocaine, choline, diethanolamine, ethylenediamine, meglumine (N-methylglucamine), and procaine.

The tetracycline compounds mentioned above, especially doxycycline and minocycline, are unexpectedly effective in preventing ARDS when administered at a dose which has substantially no antibiotic effect.

Preferably, the tetracycline compounds have low phototoxicity, or are administered in an amount that results in a serum level at which the phototoxicity is acceptable. Phototoxicity is a chemically-induced photosensitivity. Such photosensitivity renders skin susceptible to damage, e.g. sunburn, blisters, accelerated aging, erythemas and eczematoid lesions, upon exposure to light, in particular ultraviolet light. The preferred amount of the tetracycline compound produces no more phototoxicity than is produced by the administration of a 40 mg total daily dose of doxycycline.

Some antibiotic tetracyclines having low phototoxicity include, for example, minocycline and tetracyline.

Some non-antibiotic tetracyclines having low phototoxicity include, but are not limited to, tetracycline compounds having the general formulae:

### Structure K

wherein: R7, R8; and R9 taken together in each case, have the following meanings:

| R7 | R8 | R9 |
|---|---|---|
| hydrogen | hydrogen | amino |
| hydrogen | hydrogen | palmitamide |
| hydrogen | hydrogen | dimethylamino |
| and | | |

| | |
|---|---|
| STRUCTURE L | STRUCTURE M |
| STRUCTURE N | STRUCTURE O |

wherein: R7, R8, and R9 taken together in each case, have the following meanings:

| R7 | R8 | R9 |
|---|---|---|
| hydrogen | hydrogen | acetamido |
| hydrogen | hydrogen | dimethylaminoacetamido |
| hydrogen | hydrogen | nitro |
| hydrogen | hydrogen | amino |
| and | | |

STRUCTURE P

wherein: R8, and R9 taken together are, respectively, hydrogen and nitro.

The tetracycline compounds may, for example, be administered systemically. For the purposes of this specification, “systemic administration” means administration to a human by a method that causes the compounds to be absorbed into the bloodstream.

For example, the tetracyclines compounds can be administered orally by any method known in the art. For example, oral administration can be by tablets, capsules, pills, troches, elixirs, suspensions, syrups, wafers, chewing gum and the like.

Additionally, the tetracycline compounds can be administered enterally or parenterally, e.g., intravenously; intramuscularly; subcutaneously, as injectable solutions or suspensions; intraperitoneally; or rectally. Administration can also be intranasally, in the form of, for example, an intranasal spray; or transdermally, in the form of, for example, a patch.

For the pharmaceutical purposes described above, the tetracycline compounds of the invention can be formulated per se in pharmaceutical preparations optionally with a suitable pharmaceutical carrier (vehicle) or excipient as understood by practitioners in the art. These preparations can be made according to conventional chemical methods.

In the case of tablets for oral use, carriers which are commonly used include lactose and corn starch, and lubricating agents such as magnesium stearate are commonly added. For oral administration in capsule form, useful carriers include lactose and corn starch. Further examples of carriers and excipients include milk, sugar, certain types of clay, gelatin, stearic acid or salts thereof, calcium stearate, talc, vegetable fats or oils, gums and glycols.

When aqueous suspensions are used for oral administration, emulsifying and/or suspending agents are commonly added. In addition, sweetening and/or flavoring agents may be added to the oral compositions.

For intramuscular, intraperitoneal, subcutaneous and intravenous use, sterile solutions of the tetracycline compounds can be employed, and the pH of the solutions can be suitably adjusted and buffered. For intravenous use, the total concentration of the solute(s) can be controlled in order to render the preparation isotonic.

The tetracycline compounds of the present invention can further comprise one or more pharmaceutically acceptable additional ingredient(s) such as alum, stabilizers, buffers, coloring agents, flavoring agents, and the like.

The tetracycline compound may be administered intermittently. For example, the tetracycline compound may be administered 1-6 times a day, preferably 1-4 times a day.

Alternatively, the tetracycline compound may be administered by sustained release. Sustained release administration is a method of drug delivery to achieve a certain level of the drug over a particular period of time. The level typically is measured by serum concentration. Further description of methods of delivering tetracycline compounds by sustained release can be found in the patent application, “Controlled Delivery of Tetracycline and Tetracycline Derivatives,” filed on Apr. 5, 2001 and assigned to CollaGenex Pharmaceuticals, Inc. of Newtown, Pa. The aforementioned application is incorporated herein by reference in its entirety. For example, 40 milligrams of doxycycline may be administered by sustained release over a 24 hour period.

The tetracycline compounds are prepared by methods known in the art. For example, natural tetracyclines may be modified without losing their antibiotic properties, although certain elements of the structure must be retained. The modifications that may and may not be made to the basic tetracycline structure have been reviewed by Mitscher in The Chemistry of Tetracyclines, Chapter 6, Marcel Dekker, Publishers, New York (1978). According to Mitscher, the substituents at positions 5-9 of the tetracycline ring system may be modified without the complete loss of antibiotic properties. Changes to the basic ring system or replacement of the substituents at positions 1-4 and 10-12, however, generally lead to synthetic tetracyclines with substantially less or effectively no antibiotic activity.

Further methods of preparing the tetracycline compounds are described in the Examples disclosed generically or specifically in co-pending U.S. patent application Ser. Nos. 09/573,654 filed on May 18, 2000 or 10/274,841 filed on Oct. 18, 2002, which are herein incorporated by reference.

In an additional embodiment, the present invention provides a method for preventing ARDS precipitated by inhalation of toxic gases. This form of ARDS is not induced by microbes. The toxic gases may be any type of noxious gas, including for example, smoke, industrial fumes and pollutants.

The method comprises the administration of a tetracycline compound, as described above. That is, the method involves the administration of a tetracycline compound before the onset of ARDS. Preferably, the tetracycline compound is administered shortly following inhalation of the toxic gas. For example, the tetracycline compound can be administered about one hour after inhalation.

### EXAMPLES

### Example 1

### Prophylactically-Administered COL-3 in a Rat Model of Sepsis-Induced ARDS

### Methods

Surgical Procedure: Male Sprague-Dawley rats weighing between 250-300 g were acclimatized to the laboratory environment for one week prior to surgery. Free access to food and water was available for this time period. Rats were anesthetized with intraperitoneal (IP) Ketamine (90 mg/kg)/Xylazine (10 mg/kg ). Sepsis was produced using a modification of the cecal ligation and puncture (CLP) technique described by Chaudry et al. After the abdominal fur was shaved, a 2 cm midline incision was made through the skin and peritoneum. The cecum was identified and withdrawn through the incision. The avascular portion of the mesentery was sharply incised and the cecum was ligated just below the ileocecal valve with a 3-0 silk suture, so that intestinal continuity was maintained. Using an 18 gauge needle, the cecum was perforated in two locations on the antimesenteric surface and was gently compressed until feces were extruded to ensure patency of the holes. The bowel was then returned to the abdomen and the incision was closed in 2 layers using 3-0 Prolene™ for the muscle and 2-0 silk for the skin. Each rat received 10 cc physiological saline subcutaneously immediately after the procedure and at 12 and 24 hours post-surgery. The rats were allowed to recover with water and food provided ad libitum throughout the remainder of the study.

Experimental Protocol: Rats were randomly divided into 5 groups: GROUP 1) Sham CLP+2% solution of carboxymethylcellulose (CMC; vehicle for COL-3) in saline-midline laparotomy with cecum exposed and mesentery sharply incised plus oral gavage at the time of surgery with CMC (n=6); GROUP 2) Sham CLP+COL-3 (Collagenex Pharmaceutical, Newtown, Pa.)-midline laparotomy with cecum exposed and mesentery sharply incised plus oral gavage at the time of surgery with COL-3 (30 mg/kg, n=6); GROUP 3) CLP+CMC-midline laparotomy with CLP plus oral gavage at time of surgery with CMC (n=10); GROUP 4) CLP+COL-3 single dose [SD]-midline laparotomy with CLP plus oral gavage at the time of surgery with COL-3 (30 mg/kg, n=9); GROUP 5) CLP+COL-3 multiple dose [MD]-midline laparotomy with CLP plus oral gavage at the time of surgery and at 24 hours post CLP with COL-3 (30 mg/kg each administration for a total dose of 60 mg/kg, n=15). Rats were followed for 168 hours (7 days) with survival defined as hours post-CLP and survival time of each rat recorded. Rats were sacrificed at 168 hours or immediately following death. At necropsy, the left lung was excised and its bronchus cannulated. The lung was inflated to a pressure of 4 cmH2O with 10% formalin. The cannula was clamped and the lung stored in formalin at room temperature for 24 hours. The tissue was blocked in paraffin and serial sections made for staining with hematoxylin and eosin. Additionally, the remaining paraffin section of fixed lung was used for immunohistochemical determination of MMP-2 and MMP-9.

Histology: The lung tissue in each slide preparation was evaluated without knowledge of the treatment group from which it came. The slides were reviewed at low magnification for an overview to exclude sections containing bronchi, connective tissue, large blood vessels, and areas of confluent atelectasis, so that only regions reflecting the degree and stage of parenchymal injury would be evaluated. The areas of the slides which were not excluded were assessed at high magnification (400×) in the following manner. Five high power fields (HPF) were randomly sampled. Features of 1) alveolar wall thickening 2) intra-alveolar edema fluid and 3) number of neutrophils were noted in each of the 5 HPF. Specifically, alveolar wall thickening, defined as greater than two cell layers thick, was graded as “0” (absent) or “1” (present) in each field. Intra-alveolar edema fluid, defined as homogenous or fibrillar proteinaceous staining within the alveoli, was graded as “0” (absent) or “1” (present) in each field. A total score/5HPF for alveolar wall thickening and intra-alveolar edema fluid was recorded for each animal. For example, in a given animal, if all five HPF evaluated demonstrated alveolar wall thickening and intra-alveolar edema fluid the maximum score recorded would be 5/5HPF for each criteria. The total number of neutrophils was counted in each of the five HPF's and expressed as the total number/5HPF for each animal. All data was expressed as mean±SE.

Lung tissue MMP-2 and MMP-9 levels: The levels of alveolar tissue MMP-2 and MMP-9 was assessed by immunohistochemical analysis as described elsewhere. Briefly, four micrometer formalin fixed paraffin sections were treated with xylene to remove paraffin and hydrated. The paraffin sections were treated with 0.4% pepsin for 45 minutes at +37° C. For immunostaining VECTASTAIN™ Rabbit ABC Elite Kit (Vector Laboratories, Burlingame, Calif.) was used according to manufactures instructions. The endogenous peroxidase activity was blocked by incubation for 30 minutes with 0.6% H2O2 in methanol. The nonspecific binding sites were blocked by incubation with normal goat serum (1:50 in 2% Bovine Serum Albumin (BSA) in PBS for 3 hours. The sections were incubated for 1.5 hours at +37° C. and thereafter overnight (17 hours) at +4° C. with polyclonal anti-human MMP-2 (39) or monoclonal anti-rat MMP-9 antibodies (1:100 in 1% BSA in PBS) (MAB 13421, Chemicon, Temecula, Calif.). Following incubation with biotinylated anti-rabbit/anti-mouse immunoglobulin G (1:250 in 0.1% BSA in PBS) for 1 hour and with avidin-biotin complex (1:125 in PBS) for 30 minutes, the sections were stained with 3-amino-9-ethylcarbazole (AEC) (0.3 mg/ml in 0.05 M sodiumacetate, pH 5.5). The slides were washed three times 5 minutes in 0.01% Triton X-100 in PBS (140 mM NaCl, 2.7 mM KCL, 10 mM Na2HPO4, KH2PO4, pH 7.4) between each step. Counterstaining was done with Mayer's hematoxylin. For negative control the primary antibody was replaced by correspondent concentration of rabbit/mouse immunoglobulin G. The immunoreactivities of whole tissue specimens were semiquantified independently by two persons to 5 degrees (0=none, 1=mild, 2=moderate, 3=abundant, 4=strongly abundant immunoreactivity).

Lung Water: Representative tissue samples from the right lung were sharply dissected free of nonparenchymal tissue. Samples were placed in a dish and weighed, dried in an oven at 65° C. for 24 h and weighed again. This was repeated until there was no weight change over a 24-h period at which time the samples were determined to be dry. Lung water was expressed as a wet to dry weight ratio (W/D).

Serum COL-3 concentration: Blood samples to assess COL-3 levels were drawn from each rat at 48 hours after CLP. Plasma obtained was centrifuged at 3,100 rpm for 5 minutes and the supernatant was collected and frozen at −70° C. for subsequent analysis. To assay for in vivo concentration of COL-3, 50 μl plasma samples were incubated with 100 μl of precooled (−10° C.) precipitating solution containing acetonitrile:methanol:0.5M oxalic acid (60:30:10, v/v). The mixture was then centrifuged at 10,000 rpm for 5 minutes and the supernatant was collected for HPLC analysis. COL-3 concentration was determined by injecting 25 μl of the supernatant into the HPLC system using Supelco LC-18-DB reverse phase column and eluted with acetonitrile:methanol:0.1M oxalic acid (65:1:2.5, v/v) at a flow rate of 1 ml/min. Final concentration was quantified by UV detection with peak area integration at 350 nm. The limit of detection in this system was 0.2 μg/ml.

Statistical analysis: Survival rates were evaluated using the Kaplan and Meier method and the significance was determined by the generalized Wilcoxon method. Differences between groups were analyzed by one-way analysis of variance. When the F ratio indicated significance, a Newman-Keul test was used to identify individual differences. A p value less than 0.05 was considered significant. Correlations between COL-3 concentration and survival, COL-3 concentration and MMP-2 and MMP-9 levels and levels of MMP-2 and MMP-9 and survival were determined by simple linear regression analysis.

### Results

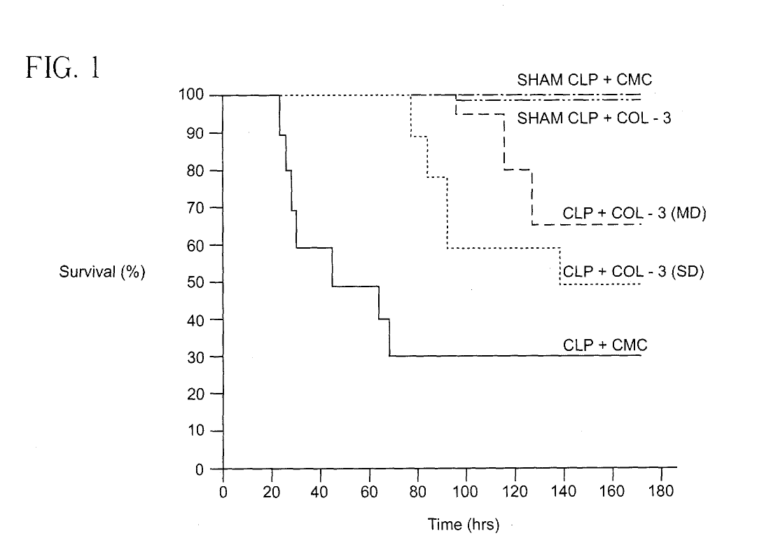

Survival: Mortality in the CLP+CMC group was 70% at 168 hours (7 days). Mortality was significantly reduced (54%) with a single prophylactic administration of COL-3 in the CLP+COL-3 (SD) group (FIG. 1). Additionally, a repeat dosing with COL-3 at 24 hours post CLP (24 hours after the first dose), further reduced mortality (33%) in the CLP+COL-3 (MD) group (FIG. 1). Animals in both Sham groups (Sham CLP+CMC and Sham CLP+COL-3) all survived.

Histology: Cecal ligation and puncture without treatment (CLP+CMC group) caused thickened and congested alveolar walls, intra-alveolar edema fluid, and marked leukocyte infiltration consistent with acute lung injury. In comparison, lung tissue from both Sham CLP groups displayed thin alveolar walls and no intra-alveolar edema fluid typical of normal lungs. These pathologic changes were reduced by the single administration of COL-3 and further attenuated by a repeat dose of COL-3 at 24 hours post CLP. The CLP+CMC group demonstrated significantly more thickened alveolar walls and intra-alveolar edema fluid as compared to both Sham CLP groups (Table VI). The number of thickened alveolar walls was significantly reduced in both the CLP+COL-3 (SD) and CLP+COL-3 (MD) groups as compared to the CLP+CMC group (Table VI). The intra-alveolar edema fluid was reduced in the CLP+COL-3 (SD) group as compared to the CLP+CMC group, but was not statistically significant. However, with the administration of a second dose of COL-3, a significant reduction in intra-alveolar edema fluid was demonstrated as compared to the CLP+CMC group (Table VI). There were no significant differences in the number of neutrophils sequestered in the lung between the CLP+CMC, CLP+COL-3 (SD), and CLP+COL-3 (MD) groups, however, all three CLP groups demonstrated significant elevations of neutrophils in the lung as compared to both Sham groups (Table VI).

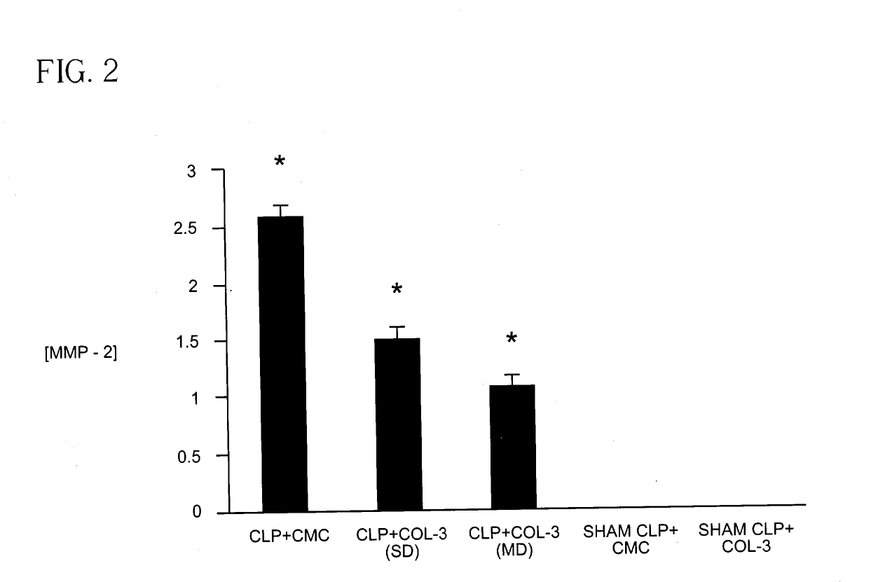

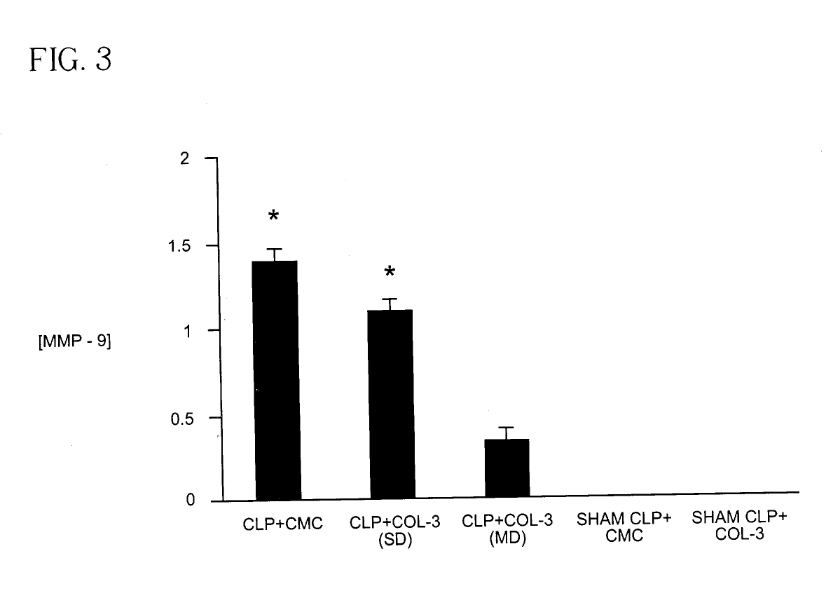

Lung tissue MMP-2 and MMP-9 levels: Representative slides of immunohistochemical staining for MMP-9 from 3 groups demonstrated varying immunoreactivity grades. Cecal ligation and puncture without treatment (CLP+CMC group) significantly increased the alveolar tissue level of both MMP-2 and MMP-9 as compared to both Sham CLP groups (FIGS. 2 and 3, respectively). COL-3 administration significantly reduced the levels of MMP-2 and MMP-9 in alveolar tissue in a dose dependent fashion (FIGS. 2 and 3, respectively). Furthermore, the repeat dose of COL-3 at 24 hours post CLP further reduced the level of MMP-9 to Sham CLP levels (FIG. 3).

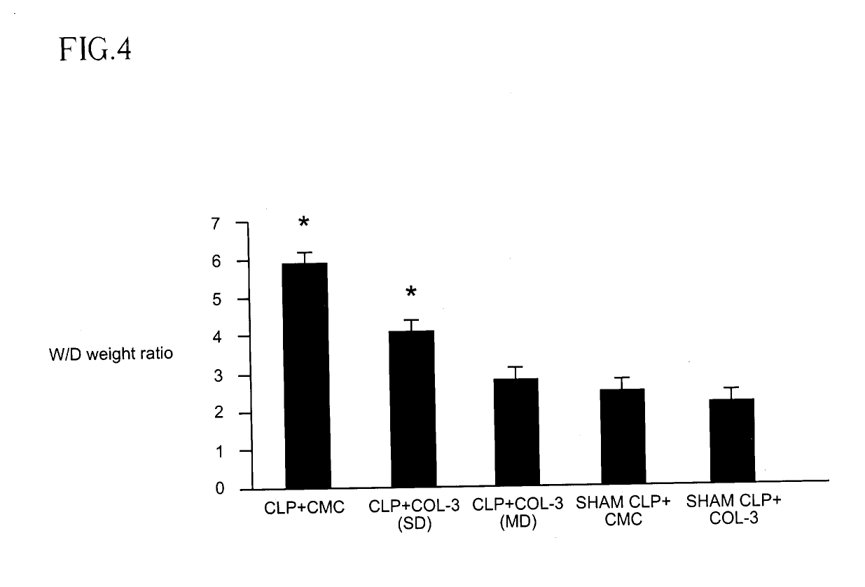

Pulmonary edema: Cecal ligation and puncture without treatment (CLP+CMC group) caused a significant increase in lung water (expressed by W/D weight ratio) as compared to both Sham CLP groups (FIG. 4). Lung water or edema was significantly reduced by the administration of COL-3 in a dose dependent manner (FIG. 4). Moreover, administration of a second dose of COL-3 at 24 hours post CLP reduced edema to Sham CLP levels (FIG. 4).

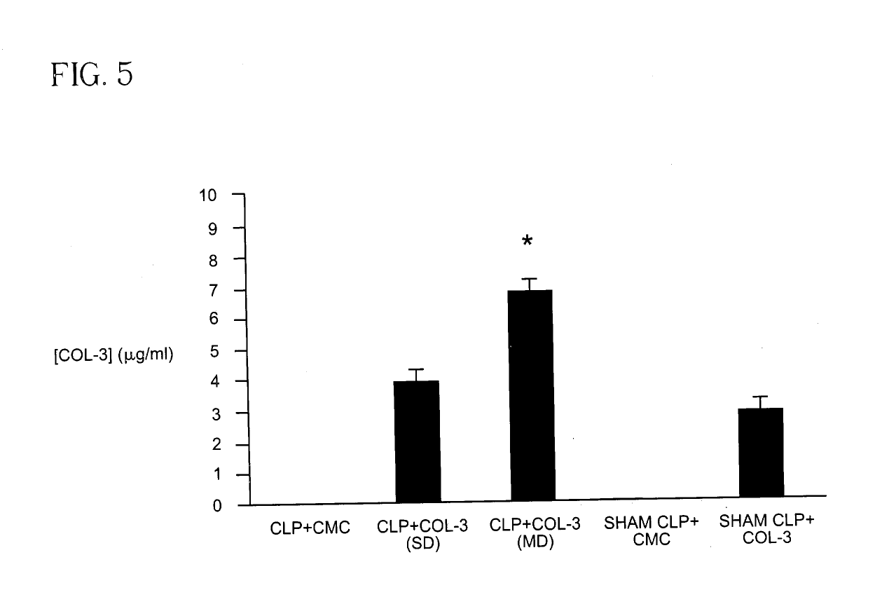

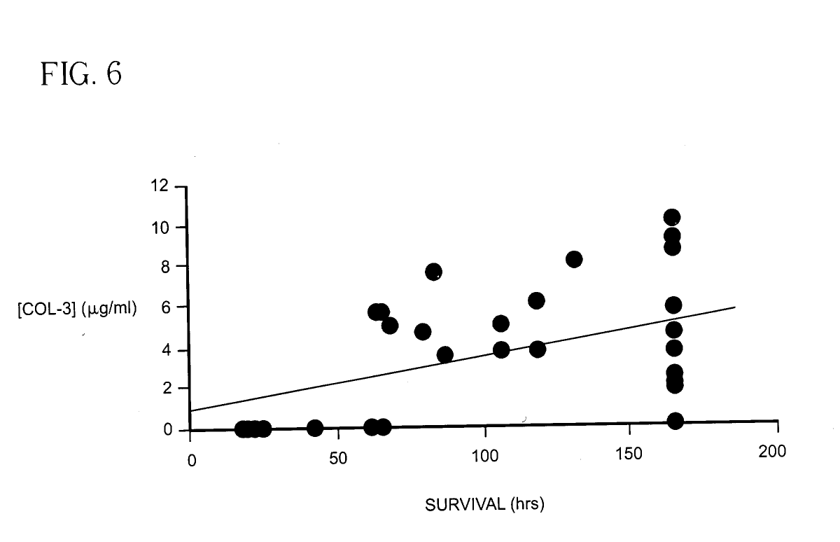

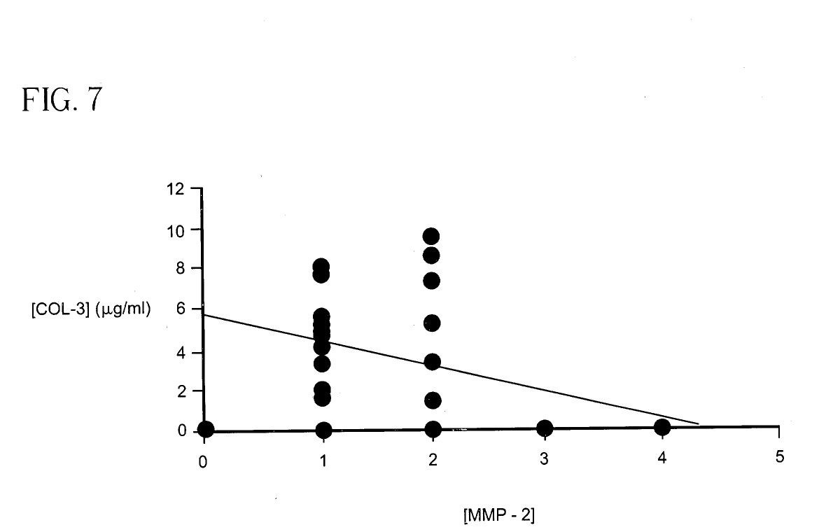

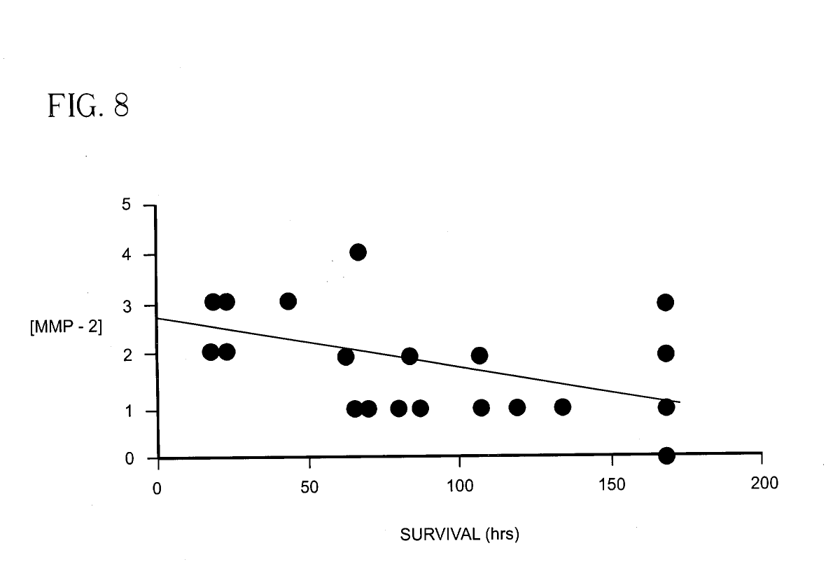

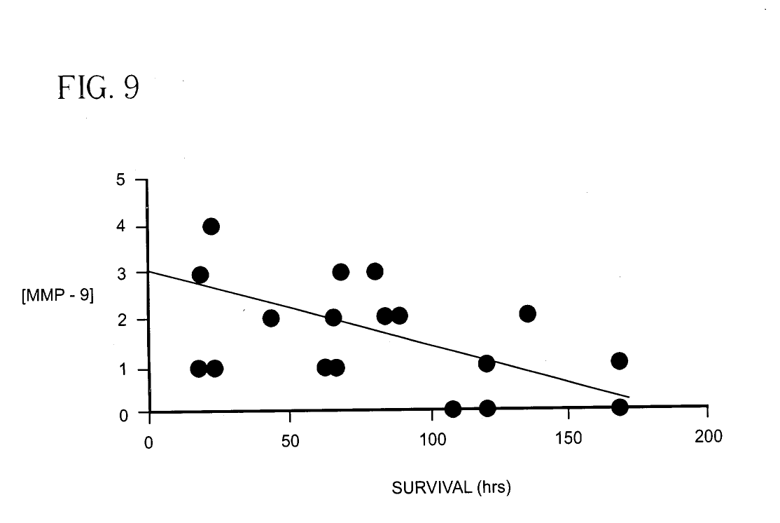

Serum COL-3 concentration: Serum concentration of COL-3 was significantly elevated at 48 hours post CLP in the CLP+COL-3 (MD) group as compared to both the CLP+COL-3 (SD) and Sham CLP+COL-3 groups (FIG. 5). A direct correlation between COL-3 concentration and improved survival was noted (FIG. 6). COL-3 concentration was inversely related to MMP-2 (FIG. 7) and MMP-9 levels, however, this did not achieve statistical significance with MMP-9 (data not shown). Furthermore, reduction of both lung tissue MMP-2 and MMP-9 levels was directly related to improved survival (FIGS. 8 and 9, respectively).

This Example demonstrates that the modified tetracycline COL-3 improves survival of rats in a dose dependent fashion in a clinically applicable model of sepsis-induced ARDS. Improvement in survival correlated with reduction of lung injury and decreased pulmonary tissue MMP-2 and MMP-9 levels.

### Example 2

### Peritoneum Fecal Clot and Clamping of the Superior Mesenteric Artery (SMA) ARDS Model Treated Prophylactically with COL-3

A sepsis-induced ARDS porcine model was developed. The placement of a fecal clot (FC) in the peritoneum was combined with clamping of the superior mesenteric artery (SMA) for 30 minutes (gut ischemia/reperfusion injury). This “two-hit” model resulted in septic shock and ARDS in 100% of the animals studied. In addition, the protocol included a 3-day termination period, due to the severity of the ARDS associated with this model and the desire to obtain clinically relevant end-point data on all animals. The protective effect of COL-3 was very dramatic. The group treated with COL-3 (SMA+FC+COL-3) demonstrated a 204% increase in PaO2/FiO2 ratio, an 80% reduction in pulmonary shunt fraction, a 64% improvement in A-a gradient, a 344% improvement in pulmonary compliance, and a 52% improvement in lung plateau pressure as compared with the SMA+FC group. In fact, all of the above parameters in the SMA+FC+COL-3 group were not statistically different from the Control group (identical surgery as the two experimental groups without placement of the fecal clot or clamping of the SMA) despite a severe bacteremia in the FC+SMA+COL-3 group (Table I). These data strongly suggest that the COL-3 treated animals would be more likely to survive than would the untreated animals. Finally, morphometric analysis of lung tissue demonstrated a 62% reduction in alveolar edema and a 94% decrease in hyline membranes with a 51% decrease in bronchoalveolarlavage fluid (BALF) protein concentration, a 87% reduction in BALF elastase activity and a 41% decrease in lung water.

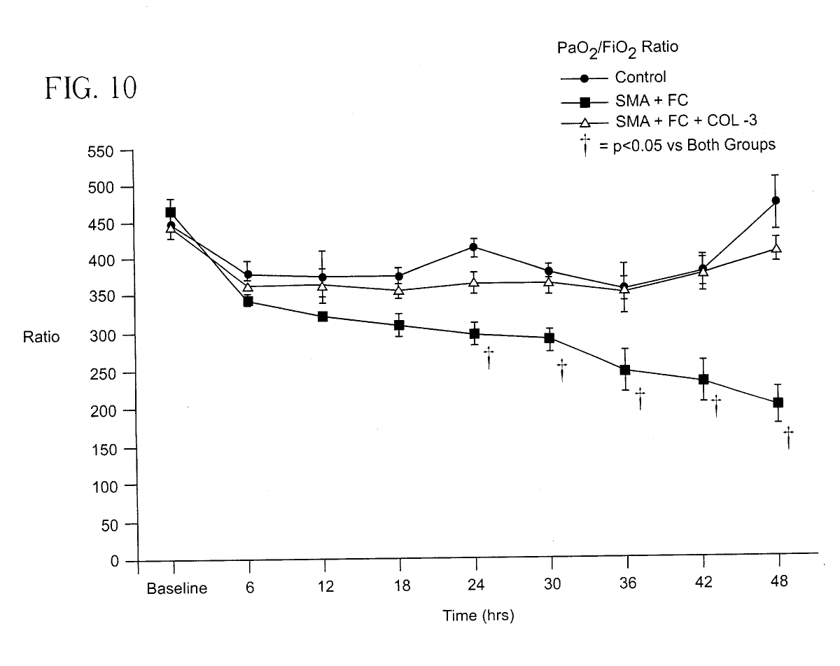

The Model:—The unique “two-hit” model caused bacteremia with or without COL-3 treatment (Table I). In fact, the COL-3 treated animals had one species of bacteria in the blood (Klebsiella Pneumoniae) not found in the non-reated group (Table I). Bacteria cultured from blood were species typical of peritonitis secondary to a perforated bowel (Table I). This “two-hit” technique caused ARDS in 100% of the pigs tested (7 for 7). All non-COL-3 treated pigs the our ARDS criteria (FiO2/PaO2 ratio less than 250) (FIG. 10) with a normal pulmonary artery wedge pressure (Table V) and were placed on mechanical ventilation within 48 hours of the surgery.

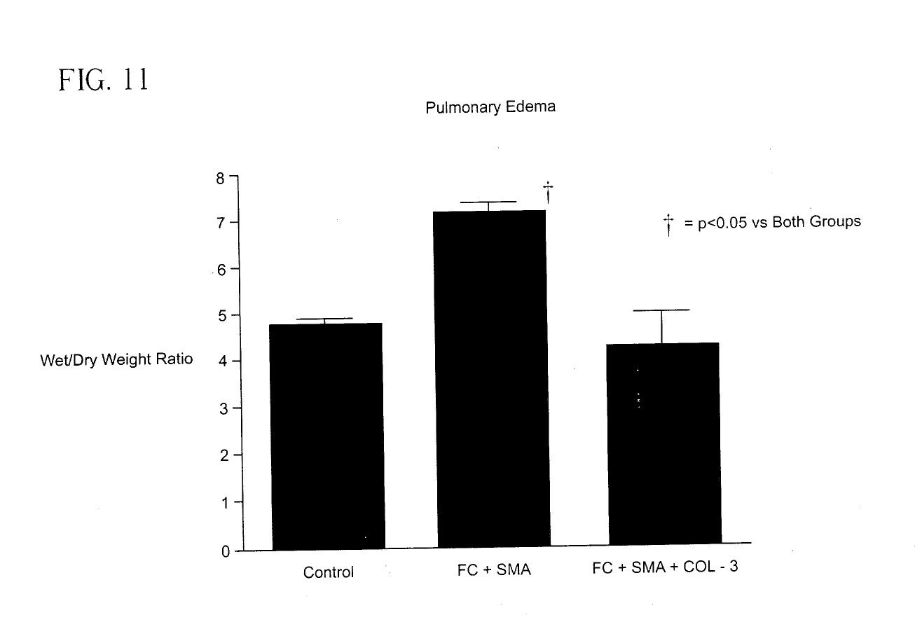

One animal met our ARDS criteria at 24 hours and four met criteria at 36 hours. ARDS was evidenced by a decrease in lung compliance (Table IV) and PaO₂/FiO₂ ratio (FIG. 10) with an increase in pulmonary shunt fraction (Table IV), pulmonary edema (FIG. 11) and histological evidence including, increased alveolar wall thickening, intra-alveolar edema and neutrophil sequestration (Table III). At necropsy two SMA+FC pigs had fulminant pulmonary edema and the lungs appeared grossly diseased as compared with the COL-3 treated lungs.

COL-3 Treatment: Blood COL-3 concentrations were as follows: Day 1=3.1±0.3, Day 2=4.9±1.0 and Day 3=3.1±1.0 μg/ml. COL-3 treatment completely prevented the development of: ARDS—evidenced by normal (i.e. not significantly different from the Control Group) lung compliance (Table IV), PaO₂/FiO₂ ratio (FIG. 10), pulmonary shunt fraction (Table IV), lung water (FIG. 11) and histological measurements (Table III). Interestingly, morphometric assessment demonstrated that the number of neutrophils sequestered in the lung was increased equally in both the SMA+FC and SMA+FC+COL-3 group as compared to the Control Group (Table II). This suggests that COL-3 will not inhibit the neutrophil's bacteriocidal properties.

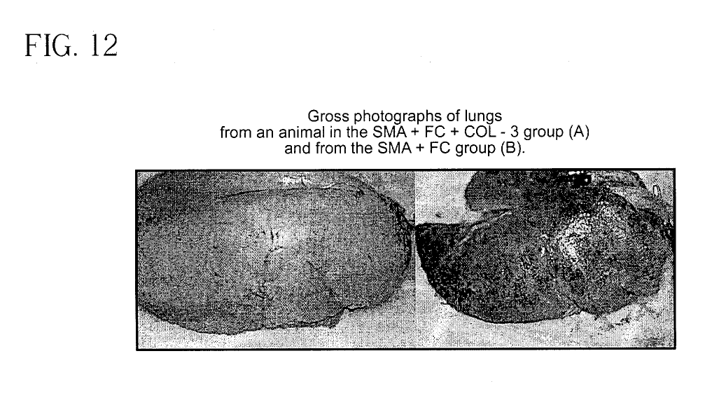

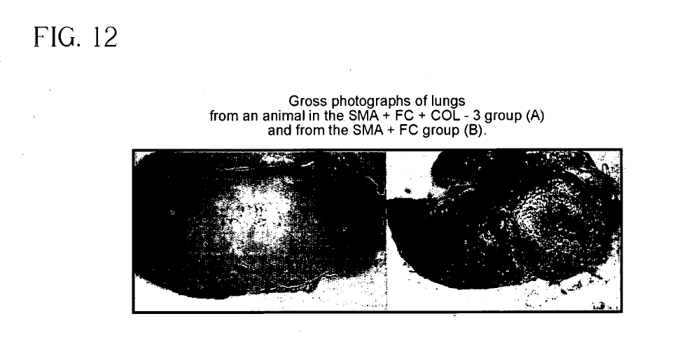

COL-3 blocked the increase in interleukin-6, IL-8, and IL-10 concentration in BALF (Table III). COL-3 also inhibited neutropil elastase (Table III) and MMP-9 in BALF. The increase in IL-10, an anti-inflammatory cytokine, only in the SMA+FC group suggests that COL-3 reduced inflammation sufficiently to prevent the release of IL-10. Interleukin-1 concentration was not significantly different in any group (Table III). These data highlight the powerful anti-inflammatory effect that COL-3 has in this very severe injury model. The near total protection of the lung with COL-3 is highlighted by the gross appearance of the lungs in each group at necropsy (FIG. 12).

A summary of the Phase I pulmonary and hemodynamic data are seen in Tables IV and V. These data demonstrate that the combined injury of a fecal clot plus clamping of the SMA causes ARDS in pigs in a time sequence and pathologic outcome analogous to ARDS in humans. COL-3 prevents sepsis-induced ARDS. The gross photograph (FIG. 12) summarizes the almost total protection offered by COL-3. Current studies have shown that COL-3 can be given as much as 12 hours following CLP and still significantly improve survival.

1. A method for preventing sepsis-induced ARDS in a mammal in need thereof, the method comprising administering to the mammal a tetracycline compound in an amount that is effective to prevent sepsis-induced ARDS but has substantially no antibiotic activity.

2. A method according to claim 1, wherein said tetracycline compound is an antibiotic tetracycline compound administered in an amount which is 10-80% of the antibiotic amount.

3. A method according to claim 1, wherein said tetracycline compound is doxycycline administered twice a day in a dose of approximately 20 mg.

4. A method according to claim 1, wherein said tetracycline compound is minocycline administered once a day in a dose of approximately 38 mg.

5. A method according to claim 1, wherein said tetracycline compound is minocycline administered twice a day in a dose of approximately 38 mg.

6. A method according to claim 1, wherein said tetracycline compound is minocycline administered three times a day in a dose of approximately 38 mg.

7. A method according to claim 1, wherein said tetracycline compound is minocycline administered four times a day in a dose of approximately 38 mg.

8. A method according to claim 1, wherein said tetracycline compound is tetracycline administered once a day in a dose of approximately 60 mg/day.

9. A method according to claim 1, wherein said tetracycline compound is tetracycline administered twice a day in a dose of approximately 60 mg/day.

10. A method according to claim 1, wherein said tetracycline compound is tetracycline administered three times a day in a dose of approximately 60 mg/day.

11. A method according to claim 1, wherein said tetracycline compound is tetracycline administered four times a day in a dose of approximately 60 mg/day.

12. A method according to claim 1, wherein said tetracycline compound is an antibiotic tetracycline compound administered in an amount which results in a serum concentration which is approximately 10-80% of the minimum antibiotic serum concentration.

13. A method according to claim 1, wherein said tetracycline compound is doxycycline administered in an amount which results in a serum concentration which is approximately 1.0 μg/ml.

14. A method according to claim 1, wherein said tetracycline compound is minocycline administered in an amount which results in a serum concentration which is approximately 0.8 μg/ml.

15. A method according to claim 1, wherein said tetracycline compound is tetracycline administered in an amount which results in a serum concentration which is approximately 0.5 μg/ml.

16. A method according to claim 2 or 12, wherein said antibiotic tetracycline compound is doxycycline, minocycline, tetracycline, oxytetracycline, chlortetracycline, demeclocycline or pharmaceutically acceptable salts thereof.

17. A method according to claim 16, wherein said antibiotic tetracycline compound is doxycycline.

18. A method according to claim 17, wherein said doxycycline is administered in an amount which provides a serum concentration in the range of about 0.1 to about 0.8 μg/ml.

19. A method according to claim 17, wherein said doxycycline is administered in an amount of 20 milligrams twice daily.

20. A method according to claim 17, wherein said doxycycline is administered by sustained release over a 24 hour period.

21. A method according to claim 20, where said doxcycline is administered in an amount of 40 milligrams.

22. A method according to claim 1, wherein said tetracycline compound is a non-antibiotic tetracycline compound.

23. A method according to claim 22, wherein said non-antibiotic tetracycline compound is:
4-de(dimethylamino)tetracycline (CMT-1),
tetracyclinonitrile (CMT-2),
6-demethyl-6-deoxy-4-de(dimethylamino)tetracycline (CMT-3),
4-de(dimethylamino)-7-chlorotetracycline (CMT-4),
tetracycline pyrazole (CMT-5)
4-hydroxy-4-de(dimethylamino)tetracycline (CMT-6),
4-de(dimethylamino)-12α-deoxytetracycline (CMT-7),
6-α-deoxy-5-hydroxy-4-de(dimethylamino)tetracycline (CMT-8),
4-de(dimethylamino)-12α-deoxyanhydrotetracycline (CMT-9), or
4-de(dimethylamino)minocycline (CMT-10).

24. A method according to claim 1, wherein said tetracycline compound has a photoirritancy factor of less than the photoirritancy factor of doxycycline.

25. A method according to claim 24, wherein said tetracycline compound has a general formula: 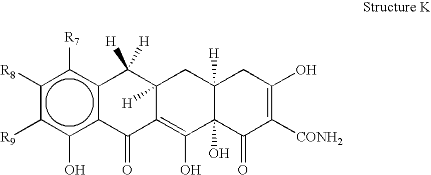
wherein R7, R8, and R9 taken together are, respectively, hydrogen, hydrogen and dimethylamino.

26. A method according to claim 24, wherein said tetracycline compound is selected from the group consisting of: 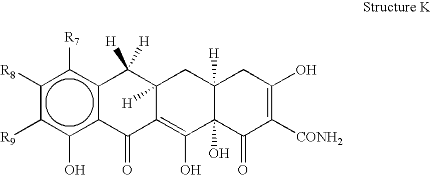
wherein R7, R8, and R9 taken together in each case, have the following meanings:

| | | |
|---|---|---|
| R7 | R8 | R9 |
| hydrogen | hydrogen | amino |
| hydrogen | hydrogen | palmitamide |
| and | | |

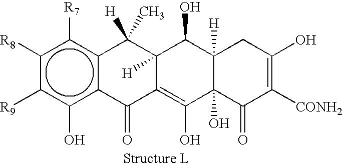

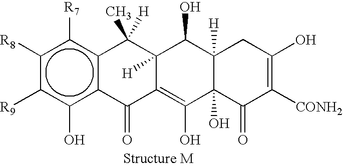

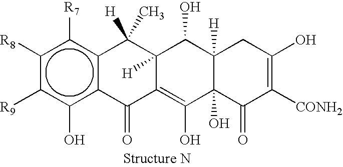

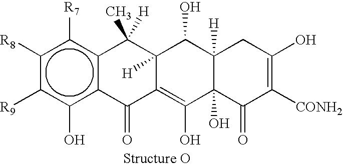

wherein R7, R8, and R9 taken together in each case, have the following meanings:

| | | |
|---|---|---|
| R7 | R8 | R9 |
| hydrogen | hydrogen | acetamido |
| hydrogen | hydrogen | dimethylaminoacetamido |
| hydrogen | hydrogen | nitro |
| hydrogen | hydrogen | amino |
| and | | |

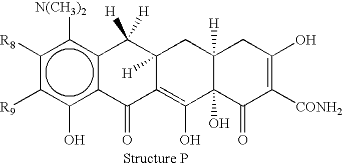

wherein R8, and R9 taken together are, respectively, hydrogen and nitro.

27. A method according to claim 1, wherein said tetracycline compound is administered systemically.

28. A method according to claim 27 wherein said systemic administration is oral administration, intravenous injection, intramuscular injection, subcutaneous administration, transdermal administration or intranasal administration.

29. A method for preventing ARDS precipitated by the inhalation of toxic gas, in a mammal in need thereof, the method comprising administering to the mammal a tetracycline compound in an amount that is effective to prevent ARDS precipitated by the inhalation of toxic gas but has substantially no antibiotic activity.
TABLE I

| | Group: | | | | | | | | | | | | | | |
|---|---|---|---|---|---|---|---|---|---|---|---|---|---|---|---|
| | Control | | | SMA + FC | | | | | | SMA + FC + COL-3 | | | | | |
| | Pig | | | | | | | | | | | | | | |
| | A | B | C | D | E | F | G | H | I | J | K | L | M | N | O |
| Day 1 | — | — | — | — | — | — | — | — | — | — | — | — | — | — | — |
| Day 2 | 5 | — | — | 2, 5, 6 | 6 | 6 | 6 | 3, 6 | 4, 6 | 4 | 1-5 | 6, 7 | 1, 3 | 5, 6 | 1, 5 |
| Day 3 | — | — | — | — | 6 | 2, 6 | 6 | 3, 6 | 4, 6 | 5, 6 | 1-5 | 6, 7 | 1, 3 | — | 1, 5 |

Individual pigs labeled A-O. Bacteria cultured in blood on Days 1-3 post surgery: Klebsiella Pneumoniae = 1, Serratia Marcescens = 2, Pseudomonas Aeruginosa = 3, Streptococci = 4, Aeromonas Hydrophila = 5, E coli = 6, Staphylococcus = 7
TABLE II
Pulmonary Histology

| | Alveolar Wall | Intra-Alveolar | |
|---|---|---|---|
| Group | Thickness | Edema | Neutrophils |
| Control | 0.6 ± 0.3 | 0.3 ± 0.3 | 103 ± 11† |
| SMA + FC | 3.7 ± 0.4† | 2.9 ± 0.3† | 221 ± 35 |
| SMA + FC + COL-3 | 1.4 ± 0.7 | 0.2 ± 0.2 | 238 ± 32 |
| Morphometric analysis of lung pathology. Wall thickness and edema | | | |
| # are expressed as the presence (1) or absence (0) of the listed | | | |
| # parameters per 5 high-powered microscopic fields. Thus the maximum | | | |
| # number each slide sampled is 5 (all fields have the presence of the | | | |
| # listed parameter). Edema is defined as homogenous or fibrillar | | | |
| # proteinaceous staining within the alveolus and Alveolar Wall | | | |
| # Thickening as greater than two cell layers thick. Neutrophils are | | | |
| # the total number in 5 high-powered fields. Data mean ± SEM. | | | |

†= p < 0.05 vs. Both Groups,
*= p < 0.05 vs. Control group.
TABLE III
Bronchoalveolar Lavage Fluid (BALF)

| GROUPS | IL-1 | IL-6 | IL-8 | IL-10 | Elastase | Protein |
|---|---|---|---|---|---|---|
| Control | 646 ± 44 | 4 ± 4* | 5 ± 2* | 0* | 12 ± 3* | 500 ± 116* |
| SMA + FC | 750 ± 168 | 1,400 ± 690 | 89 ± 46 | 53 ± 3 | 64 ± 20 | 1,353 ± 291 |
| SMA + FC + COL-3 | 622 ± 255 | 7 ± 6* | 5 ± 3* | 0* | 8 ± 2* | 663 ± 85* |

The concentration of interleukins-1, -6, -8, -10 (pg/ml), protein (ug/100 ml), and neutrophil elastase activity (μmol substrate degraded/mg protein/18 hr) in the bronchoalveolar lavage fluid. Data are mean ± SEM. * = p < 0.05 vs. SMA + FC group.
A summary of the Phase I pulmonary and hemodynamic data are seen in Tables IV and V.
TABLE IV
Pulmonary Parameters

| Var- | | | | | |
|---|---|---|---|---|---|
| iable | Group | 24 hrs | 36 hrs | 48 hrs | |
| | Control | ND | ND | 25 | ± 1 |
| Ppeak | SMA + FC | 31 (n = 1) | 36 ± 3.5 (n = 4) | 46 | ± 4.6 # |
| | SMA + FC + | 26 (n = 1) | 24 (n = 1) | 23 | ± 0.5 |
| | COL-3 | | | | |
| | Control | ND | ND | 21 | ± 1.6 |
| Pplat | SMA + FC | 26 (n = 1) | 31 ± 3.7 (n = 4) | 44 | ± 4.3 # |
| | SMA + FC + | 24 (n = 1) | 26 (n = 1) | 21 | ± 0.4 |
| | COL-3 | | | | |
| | Control | ND | ND | 2.3 | ± 0.3 |
| Rinsp | SMA + FC | 29 (n = 1) | 17 ± 4.8 (n = 4) | 47 | ± 6 # |
| | SMA + FC + | 10 (n = 1) | 7 (n = 1) | 3.6 | ± 0.6 |
| | COL-3 | | | | |
| | Control | ND | ND | 32 | ± 4.9 |
| Com- | SMA + FC | 18 (n = 1) | 15.4 ± 3 (n = 4) | 9 | ± 1.7 # |
| pliance | | | | | |
| | SMA + FC + | 26 (n = 1) | 26 (n = 1) | 31.6 | ± 2 |
| | COL-3 | | | | |
| | Control | ND | ND | 5 | ± 0.5 |
| Shunt | SMA + FC | 12 (n = 1) | 17 ± 4.5 (n = 4) | 27.7 | ± 5.2 # |
| | SMA + FC + | 7 (n = 1) | 5 (n = 1) | 5.6 | ± 0.9 |
| | COL-3 | | | | |
| | Control | ND | ND | 168 | ± 9 |
| A-a | SMA + FC | 174 (n = 1) | 168 ± 26 (n = 4) | 280 | ± 66 # |
| gradient | | | | | |
| | SMA + FC + | 144 (n = 1) | 130 (n = 1) | 100 | ± 10 |
| | COL-3 | | | | |
| Table IV. Ppeak = peak airway pressure (cm H2O); | | | | | |
| Pplat = airway plateau pressure (cm H2O); | | | | | |
| Rinsp = airway resistance to inspiratory flow (cm H2O/(l/s)); | | | | | |
| Compliance = static compliance (ml/cm H2O); | | | | | |
| Shunt = shunt fraction (%); | | | | | |
| A-a gradient = Alveolar-arterial PaO2 difference (mm Hg); | | | | | |
| ND = no data. | | | | | |
| Data are mean ± SE. | | | | | |
| * = p < 0.05 vs. Control; | | | | | |
| # = p < 0.05 vs. both Control and SMA + FC + COL-3. | | | | | |
| Note: | | | | | |
| At 24 hrs 1 out of 4 animals (n = 1) in the SMA + FC group and 1 out of 5 | | | | | |
| # animals (n = 1) in the SMA + FC + COL-3 group required mechanical | | | | | |
| # ventilation and had placement of a Swan Ganz catheter for monitoring (in | | | | | |
| # the SMA + FC group this was due to the animals clinical decline, while in | | | | | |
| # the SMA + FC + COL-3 group this was due to technical difficulty with the | | | | | |
| # arterial line). At 36 hrs 4 out of 7 animals (n = 4) in the SMA + FC group | | | | | |
| # were on mechanical ventilation with Swan Ganz catheter monitoring (all due | | | | | |
| # to animals clinical decline), and by 48 hrs all animals in the SMA + FC | | | | | |
| # group (n = 7) had required mechanical ventilation and Swan Ganz catheter | | | | | |
| # monitoring secondary to their clinical deterioration. The remainder of the | | | | | |
| # animals in the SMA + FC + COL-3 group (4 additional animals for an n = 5) | | | | | |
| # and the animals in the Control group (n = 3) were placed on the ventilator | | | | | |
| # and sacrificed at 48 hours. | | | | | |

TABLE V
Hemodynamic Parameters.

| | | | | | | |
|---|---|---|---|---|---|---|
| Variable | Group | +HL, 0 hrs | 12 hrs | 24 hrs | 36 hrs | 48 hrs |
| pH | Control | 7.43 ± .01 | 7.53 ± .07 | 7.53 ± .03 | 7.47 ± .03 | 7.52 ± .02 |
|---|---|---|---|---|---|---|
| | SMA + FC | 7.41 ± .03 | 7.51 ± .02 | 7.40 ± .02 | 7.39 ± .04 [symbol] # | 7.28 ± 0.1 [symbol] # |
| | SMA + FC + COL-3 | 7.48 ± .03 | 7.56 ± .02 | 7.54 ± .01 | 7.54 ± .02 | 7.51 ± .03 |
| PCO2 | Control | 36 ± 4.8 | 38 ± 2 | 31 ± 0.8 | 31 ± 1.2 | 30 ± 3 |
| | SMA + FC | 39 ± 3.5 | 37 ± 2.6 | 25 ± 2.2 [symbol] | 27 ± 3.1 [symbol] # | 42 ± 5 |
| | SMA + FC + COL-3 | 35 ± 3.8 | 31 ± 1.5 | 29 ± 1.2 | 30 ± 1.6 | 32 ± 2.2 |
| BE | Control | 5.3 ± .8 | 7 ± 1.0 | 5 ± 0.7 | 4.3 ± 0.6 | 5.8 ± 0.4 |
| | SMA + FC | 4.8 ± 1.1 | 10 ± 3.2 [symbol] # | 5.8 ± 2.1 | 1.2 ± 3.4 [symbol] # | −2.8 ± 3.2 [symbol] # |
| | SMA + FC + COL-3 | 3.8 ± 0.3 | 5.8 ± 0.3 [symbol] | 8.6 ± 0.8 [symbol] # | 4.8 ± 0.9 | 6.4 ± 0.6 [symbol] |
| Hgb | Control | 11.7 ± 0.3 | 13 ± 1.1 | 11 ± 1.5 | 12.3 ± 0.3 | 11.3 ± 0.3 |
| | SMA + FC | 12.2 ± 0.3 | 12.1 ± 0.4 | 12.2 ± 0.4 | 11 ± 0.3 | 10.7 ± 0.4 |
| | SMA + FC + COL-3 | 11.8 ± 0.2 | 12.1 ± 0.8 | 10.8 ± 0.5 | 10.9 ± 0.5 | 11 ± 0.4 |
| SBP | Control | 124 ± 4 | 136 ± 7.2 | 130 ± 7 | 131 ± 8 | 120 ± 10 |
| | SMA + FC | 115 ± 7.4 | 125 ± 5 | 147 ± 12 [symbol] | 99 ± 6 [symbol] # | 79 ± 6.4 [symbol] # |
| | SMA + FC + COL-3 | 135 ± 4.8 | 137 ± 4.7 | 140 ± 7.6 | 130 ± 8.2 | 130 ± 4.2 |
| DBP | Control | 80 ± 3.6 | 98 ± 6.7 | 85 ± 11.7 | 80 ± 11.2 | 90 ± 12 |
| | SMA + FC | 74 ± 5.4 | 71 ± 8.6# | 88 ± 8 | 48 ± 6.9 [symbol] # | 37 ± 4.5 [symbol] # |
| | SMA + FC + COL-3 | 82 ± 6.9 | 98 ± 4.4 | 98 ± 4.3 | 90 ± 3.4 | 93 ± 5 |
| HR | Control | 107 ± 2 | 137 ± 2.4 [symbol] | 125 ± 6.6 | 130 ± 6.1 | 117 ± 7.8 |
| | SMA + FC | 129 ± 6.5 | 156 ± 8 | 163 ± 7.7 [symbol] * | 151 ± 11 | 127 ± 14 |
| | SMA + FC + COL-3 | 109 ± 7 | 149 ± 6.7 [symbol] | 141 ± 4.5 | 133 ± 4.2 | 125 ± 7.4 |
| RR | Control | 31 ± 1.6 | 36 ± 1.6 | 35 ± 2.8 | 41 ± 1.6 | 15 ± 0 [symbol] |
| | SMA + FC | 35 ± 1.5 | 83 ± 1.6 [symbol] # | 71 ± 11 [symbol] # | 40 ± 13 | 17 ± 0.8 [symbol] |
| | SMA + FC + COL-3 | 28 ± 1.2 | 46 ± 4 | 39 ± 6.5 | 39 ± 6 | 15 ± 0 [symbol] |
| Temp | Control | 98.9 ± 0.4 | 101.3 ± 0.8 [symbol] | 100.7 ± 0.2 [symbol] | 100 ± 0.1 [symbol] | 99.8 ± 0.1 |
| | SMA + FC | 99.9 ± 0.6 | 104.5 ± 0.2 [symbol] * | 104 ± 0.2 [symbol] * | 102 ± 1.4 [symbol] * | 97.4 ± 0.7 [symbol] * |
| | SMA + FC + COL-3 | 99.6 ± 0.4 | 105 ± 0.4 [symbol] * | 103.9 ± 0.3 [symbol] * | 103.6 ± 0.5 [symbol] * | 102.2 ± 0.5 [symbol] * |
| Ppa | Control | ND | ND | ND | ND | 18 ± 0.3 |
| | SMA + FC | ND | ND | 23 (n = 1) | 25 ± 3.4 (n = 4) | 27.5 ± 4# |
| | SMA + FC + COL-3 | ND | ND | 21 (n = 1) | 15 (n = 1) | 16.6 ± 1.4 |
| Ppw | Control | ND | ND | ND | ND | 7.3 ± 1.3 |
| | SMA + FC | ND | ND | 9 (n = 1) | 9.2 ± 1.1 (n = 4) | 10.3 ± 1.8 |
| | SMA + FC + COL-3 | ND | ND | 8 (n = 1) | 8 (n = 1) | 8.2 ± 1.3 |
| CO | Control | ND | ND | ND | ND | 6.2 ± 1.4 |
| | SMA + FC | ND | ND | 6.7 (n = 1) | 6.5 ± 1.2 (n = 4) | 3.6 ± 0.5# |
| | SMA + FC + COL-3 | ND | ND | 6.8 (n = 1) | 7.3 (n = 1) | 7.6 ± 1.3 |
| Table V. BE = base excess; Hgb = hemoglobin; SBP = systolic blood pressure (mm Hg); DBP = diastolic blood pressure (mm Hg); HR = heart rate (beats/min); RR = respiratory rate (resp/min); Temp = temperature (° F.); Ppa = mean pulmonary artery pressure (mm Hg); Ppw = pulmonary wedge pressure (mm Hg); CO = cardiac output (L/min); ND = no data. Data are mean ± SE. * = p < 0.05 vs. Control; [symbol] = p < 0.05 | | | | | | |
| # vs. BL; # = p < 0.05 vs. both Control and SMA + FC + COL-3. Note: At 24 hrs 1 out of 7 animals (n = 1) in the SMA + FC group and 1 out of 5 animals (n = 1) in the SMA + FC + COL-3 group required mechanical ventilation and had placement of a Swan Ganz catheter for monitoring (in the SMA + FC group this was due to the animals clinical decline, while in the SMA + FC + COL-3 group this was due to technical difficulty | | | | | | |
| # with the arterial line). At 36 hrs 4 out of 7 animals (n = 4) in the SMA + FC group were on mechanical ventilation with Swan Ganz catheter monitoring (all due to animals clinical decline), and by 48 hrs all animals in the SMA + FC group (n = 7) had required mechanical ventilation and Swan Ganz catheter monitoring secondary to their clinical deterioration. The remainder of the animals in the SMA + FC + COL-3 group (4 additional animals for an | | | | | | |
| # n = 5) and the animals in the Control group (n = 3) were placed on the ventilator and sacrificed at 48 hours. | | | | | | |

TABLE VI
Histological grading of alveolar wall thickening, intra-alveolar
edema formation, and number of neutrophils.

| | ALVEOLAR WALL | INTRA-ALVEOLAR | |
|---|---|---|---|
| | THICKENING/ | FLUID(EDEMA)/ | NEUTROPHILS/ |
| | 5 HPF | 5 HPF | 5 HPF |
| CLP + CMC | 4.4 ± .62* | 3.1 ± .91 [symbol] | 382.3 ± 43.1¶ |
| CLP + COL-3 (SD) | 2.0 ± 1.0 | 1.7 ± 1.0 | 391.5 ± 50.1¶ |
| CLP + COL-3 (MD) | 1.8 ± .55 | 1.0 ± .57 | 361.7 ± 60.9¶ |
| SHAM CLP + CMC | 0 | 0 | 170.3 ± 38.9 |
| SHAM CLP + COL-3 | 0.5 ± 0.5 | 0 | 195.8 ± 38.8 |

CLP = cecal ligation and puncture;
CMC = carboxymethylcellulose (vehicle);
COL-3 = chemically modified tetracycline;
SD = single dose;
MD = multiple dose;
HPF = high power fields.
*= p < 0.01 vs all groups [symbol]
= p < 0.02 vs CLP + COL-3 (MD) and both Shams
¶= p < 0.04 vs both Shams
